(19) United States
(12) Patent Application Publication
(10) Pub. No.: US 2004/0093170 A1
(43) Pub. Date: May 13, 2004

(54) Method for determining pharmaceutically active substances

(76) Inventors: Soheila Anzali, Seeheim-Jugenheim (DE); Gerhard Barnickel, Darmstadt (DE); Bertram Cezanne, Morfelden-Walldorf (DE); Michael Krug, Bickenbach (DE); Joachim Marz, Gross-Gerau (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/468,375

(22) PCT Filed: Feb. 13, 2002
(86) PCT No.: PCT/EP02/01472

(30) Foreign Application Priority Data
Feb. 22, 2001 (DE) 101 08 590.7

(51) Int. Cl. G06F 19/00; C12Q 1/00; C12Q 1/68; A01N 37/18; A01N 43/04

(52) U.S. Cl. 702/27; 435/4; 435/6; 514/2; 514/44

(57) ABSTRACT

The present invention relates to a method for determining pharmaceutically active substances with the aid of test series, in which the action of a large number of different substances on one or more biological targets is tested and a hit list of those substances which actually or apparently have shown a pharmaceutical action on at least one biological target is prepared, whereupon a selection is made from the hits for more detailed investigations of pharmaceutical activity and/or applicability. In order to provide a method of the type stated at the outset, by means of which the success rate can be significantly increased, the following features are proposed: a) Determination of the chemical structure of the substances in the hit list, b) splitting of the structure of the substances into individual components or fragments, c) classification of the substances into groups having in each case identical and/or virtually identical or similar fragments, d) evaluation of the pharmacological activity of the various groups, e) comparison of the pharmacological activity of the various groups and f) selection of substances from those groups which, on the basis of the evaluation and of the comparison according to steps d and e, have the highest activity.

The present invention relates to a method for determining pharmaceutically active substances with the aid of test series, with which the action of a large number of different substances on one or more biological targets is tested and a hit list of those substances prepared which actually or apparently have shown a pharmaceutical action on at least one biological target, whereupon a selection is made among the hits for more detailed investigations of the pharmaceutical activity and/or applicability.

Corresponding test series have been carried out recently in particular in the form of “High Throughput Screening” (HTS). The typical collection of substances or test compounds of an HTS experiment comprises, for example, one million different molecules which are stored on microtitre plates in the form of DMSO solutions. The HTS system is composed of a number of apparatuses which are physically connected to one another by a transport mechanism which, generally in the form of a robot arm, carries out the various tasks within the HTS experiment. The critical point for a productive and reliable HTS experiment is the ability of the robot system to carry out the corresponding functions reproducibly and in a stable manner with the given requirements and within a predetermined time. With a larger number of experimental steps, i.e. with more complicated test arrangements, more apparatuses are accordingly involved, so that the risk of failures and incorrect results as a result of not complying with specified boundary conditions in any of the steps of the experiment increases.

Furthermore, the method of determining the biological response of the experimental test system or of the targets has various sources of errors, for example in so far as a response or reaction is disturbed or masked by the molecular properties of individual test molecules.

A further difficulty is the concentration dependence of the biological response. A concentration which is too high or too low can in each case both lead to false positive and to false negative results, i.e. the relevant substance is characterized either as a hit or not a hit, whereas another result would be obtained with a different, actually realizable concentration. The actual effective concentration may change in the course of an experiment through a variety of factors, such as, for example, exceeding the solubility, or chemical reaction with excipients used. The consequence of all these sources of errors is evident from the fact that the hit list finally resulting from the biological response of the system contains a large number of false positive results, i.e. substances characterized as being pharmaceutically active for which in reality such activity cannot be confirmed later on, whereas a large number of substances are presumed to be pharmaceutically inactive or pharmaceutically insufficiently active which possibly nevertheless have a high pharmaceutical potential.

Whereas the main problem of HTS experiments a few years ago was that the detection of the biological response required a very long time, this aspect is now of minor importance owing to more recent developments.

On the other hand, it is still very labour-intensive and time-consuming to identify those substances or molecules which actually have a considerable pharmaceutical action and to verify this in detail, particularly active and usable substances and molecules which are suitable for further development to a medicament often also being referred to as “control molecules”. Usually, a procedure is adopted in which those substances which actually or apparently show the strongest biological response are selected from the hit list in order subsequently to investigate these substances in more detail on the basis of various criteria and conditions.

The extensive testing of a pharmaceutically active substance for its actual suitability as a medicament may take weeks or months. A clear indication of all hits of an HTS experiment is accordingly impossible for practical reasons. Of course, a large number of substances whose biological response is below an arbitrarily specified limit are therefore not considered.

This is evident from the consideration of a typical HTS experiment in which, for example, 250,000 different compounds are investigated. Even if the hit rate is only 0.5%, this delivers 1250 different compounds, of which only a small part can be selected for the more detailed investigation, owing to the scope of the further tests.

The selection criteria for selecting the best possible test candidates are therefore of decisive importance for the success rate, i.e. the discovery of control molecules for the more detailed investigation of selected substances from the hit list.

Starting from this prior art, it is therefore the object of the present invention to provide a method of the type stated at the outset by means of which the success rate can be significantly increased.

This object is achieved if the method comprises the following steps:

a) Determination of the chemical structure of the substances in the hit list,

b) splitting of the structure of the substances into individual components or fragments,

c) classification of the substances into groups having in each case identical and/or virtually identical or similar fragments,

d) evaluation of the pharmacological activity of the various groups,

e) comparison of the pharmacological activity of the various groups,

f) selection of substances from those groups which, on the basis of the evaluation and of the comparison according to steps d and e, have the highest activity.

According to the invention, no evaluation of the hits is therefore initially carried out according to the intensity of the biological response, but rather the apparently pharmaceutically active substances are first determined with respect to their chemical structure, and this structure is investigated for corresponding fragments, substances which have a fragment in common being assigned to a group defined by this fragment.

Although an evaluation of the pharmaceutical activity of the substance is then also carried out, the pharmaceutical action of an individual substance or the intensity of the biological response of an individual substance is no longer the decisive criterion as to whether this substance will be investigated in more detail, but rather an evaluation of the total group to which the relevant substance belongs is performed and it is only the result of this evaluation that decides whether the group as a whole or individual substances from this group will be selected for more detailed investigations of the pharmaceutical activity.

The fragmentation can be effected in various ways, the expedient representation of the fragments being of primary importance. Thus, a preferred method is one in which the chemical structures are represented in linear form as a character chain so that identical fragments can be characterized by an identical linear chain of predetermined structural elements and predetermined length. However, methods which describe chemical structures by numerical parameters (e.g. molecular indices) can also be used.

A precondition for the formation of the groups is the discovery of a reversible unique relationship between a defined fragment and its linear or numerical representation. If the selected method meets this requirement, the assignment to groups is effected substantially by a combination of identical representations.

However, the selection of characteristic fragments which possibly dominate or influence the pharmaceutical action plays an important role. This is preferably effected by using at least one, and preferably more, of the following steps:

a) Removal of all simple substitutions on a molecular skeleton,

b) extraction of the associated ring systems of a molecule,

c) extraction of all simple ring structures of a molecule,

d) extraction of all molecular moieties present in the form of a chain,

e) selection of topological paths within a molecule with predetermined length,

f) taking into account concentric atomic neighbourhood spheres around selected atoms,

g) combination or permutation of all substituents of a molecular skeleton,

h) determination of the sets of atoms linked via topological paths,

i) fragmentation by breaking of bonds of defined chemical groups,

j) taking into account sets of torsional angles of atoms, and

k) selection of existing predefined functional groups as a fragment.

In the comparison of the various structures for corresponding fragments, substances are therefore preferably checked for corresponding ring structures or other characteristic structural elements on the basis of the above criteria and can, for example, also be assigned to the same group when different atoms (or ions) or molecules are present at substitution sites, for example when a carbon atom is replaced by a nitrogen atom in an aromatic ring.

The approach of sorting by characteristic fragments moreover offers the previously unknown or unused possibility of correcting false negative test results, i.e. of selecting from the predominant number of substances which were initially excluded as negative by the test, and adding to the relevant group, those which have characteristic structural fragments in common with the substances characterized as hits. It is of course true in particular for those substances tested as negative which possibly have a plurality of other properties in common with the substances of a group which have tested positive, e.g. a similar molecular weight or similar active groups. However, substances which have tested negative and which have a fragment which characterizes a group of substances which have tested positive should be added to this group only when the total number of such substances which have tested negative is not too large in relation to the total number of members of the group which have tested positive. In fact, if the number of substances which have tested negative is larger than or even substantially larger than the number of substances which have tested positive and which have this fragment in common as a group, it is probably an indication that the relevant substances of this group tested false positive, unless they have a further fragment which also makes them a member of another group. In this case, the group with the relevant fragments can nevertheless be excluded since a large number of substances which tested negative have the same fragment, so that the biological action of the substances which tested positive is either due to an error in the test procedure or is due to the fact that another fragment of these substances resulted in the positive response of the biological system.

Since the substances optionally can also be simultaneously assigned to different groups if they have many corresponding fragments which they possess in common with other active substances, in this way only those substances which have no further fragments in common with other substances which have tested positive are excluded.

In particular, the bioisosteric molecules of the test series should be included in the substance groups which were formed on the basis of common fragments. Bioisosteric is understood as meaning those compounds or molecules which, in spite of exchange of an atom or of a group of atoms, retain a considerable pharmacological action. Of course, such bioisosteric molecules have a high degree of similarity with one another. This means that a specific pharmacological action can be predicted for a molecule with high probability if it is known that a chemically and structurally very similar molecule has just this action.

As already mentioned, this method also permits the exclusion of false positive results although a strongly positive biological response may be present. If it is in fact found, on inclusion of the substances which have tested negative, that a group of substances which have tested positive and have a common structural element has a very large number of substances among the substances which have tested negative or only a very large number of substances which tested negative and have the same fragment were present in the test series, it is highly probable that even the substances of this group which tested positive will exhibit no pharmaceutical action or only a very slight pharmaceutical action on more detailed investigation.

A further aspect of the present invention which can be used in addition to the fragmentation and optionally also completely independently of the fragmentation as a novel selection criterion is the particular method of evaluation of the substance as a candidate for a further detailed investigation. This provides in particular the evaluation of a whole set of parameters, in each case separately and preferably also in combination with one another.

The most important evaluation criteria include molecular weight, molecular size, ionization, basicity, acidity, lipophilicity/ amphiphilicity, solubility, octanol/water partition coefficient, number of hydrogen bridge acceptors and donors, stability in gastric and intestinal fluid, permeability values for various cell types and toxicity.

In addition, there is of course a number of further substance properties which in certain circumstances are important criteria for the selection of suitable candidates for the further investigation. These properties are shown in Table 1 below, under the heading “Substance properties for scoring”.

TABLE 1
Substance properties for scoring

Molecular size/molecular surface/molecular conformation and associated
sizes
Ionization and basicity/acidity (e.g. pKa)
Lipophilicity/ampiphilicity profiles
Solubility profiles
Dissolution profiles
Melting point/boiling point
Particle size
Reactivity
Synthesizability and derivatizability
Analytical purity
PASS profiles
Number and strength of hydrogen bridge donors/acceptors
Molecular properties calculated via incremental systems
Presence of functional groups of known drugs
Photochemical stability
Colour and optical absorbance, extinction
Hit properties in various HTS experiments
Stability in gastric/intestinal fluid or in plasma or chemical stability
Active transport
Permeability values Caco-2/TC-7/endothelial cells
Metabolic stability in hepatic microsomes/Caco-2/hepatic cells
Data from inhibition tests on cytochromes
Plasma protein binding
Data from animal experiments
Pharmacokinetic data
Receptor binding data
Receptor selectivity (split)
Functional data
Toxicological data/warnings
Adverse reactions
Patentability/novelty
Investments/costs
Expected sales/profits

Particularly preferably, the evaluation is performed on the basis of a preselected set of the properties stated in the table, which are expediently standardized for this purpose. A possible function which represents the corresponding properties in standardized form would be, for example, a function of the form

$${S}_{x} = \frac{x - {x}_{min}}{{x}_{max} - {x}_{min}} .$$

Of course, there is also a large number of further functions by means of which the relevant properties could, if required, be represented in standardized form.

For example, the sum of the evaluations of the individual properties is used for the overall evaluation of a substance, the individual summands preferably also being weighted. For the evaluation of a group of substances which have the same fragment, the sum of the evaluations of the individual substances is used. This sum is of course the greater the more numerous the members of such a group, which is also entirely desirable since the relevant group evidently has a particularly high pharmaceutical potential. It is of course also possible for the sum of the individual evaluations of the substances of a group to be divided by the number of members of this group in order to obtain information about the average strength or the average potential of the individual substances of this group, this being expedient particularly when the group was extended to include members which tested negative.

Individual property values can also be multiplied by the sum of the other properties or by the product of other properties, as factors. Such factorization of the properties is possible in particular when a property represents an absolute exclusion criterion, such as, for example, a high toxicity. If the toxicity is represented on a standardized scale, i.e. for example ranging from 0 to 1, this property can be taken into account in the form of a factor (1 minus toxicity), since this factor is equal to zero if the toxicity is maximum, i.e. is 1, so that this factor then dominates all other evaluations.

Of course, a large number of mathematical functions and methods can be used for obtaining a practically expedient evaluation of substances and also whole groups of substances, the special feature of the method according to the invention being the use of uniform, standardized evaluation criteria which depend only to a certain extent on the strength of the biological response, which has been used to date as an essential evaluation criterion.

All substances of the HTS experiment and their structures, in particular of the hits, are advantageously stored in digital form. This moreover has the advantage that it is even possible to include in the evaluation (group formation by fragments and evaluation) substances which were not used at all in the relevant HTS experiment but whose structural data and/or relevant parameters are possibly known and are available in private or public databases.

Further advantages, features and potential applications of the present invention are evident from the following description of an embodiment and the associated Figures.

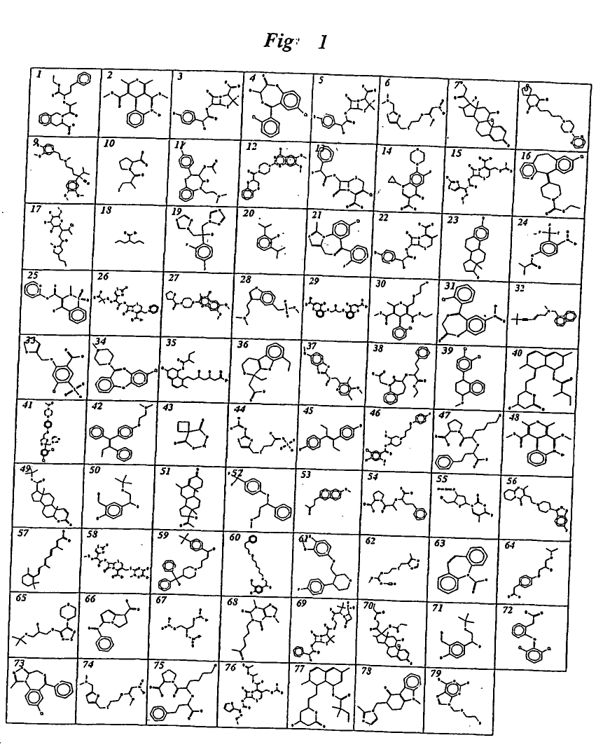

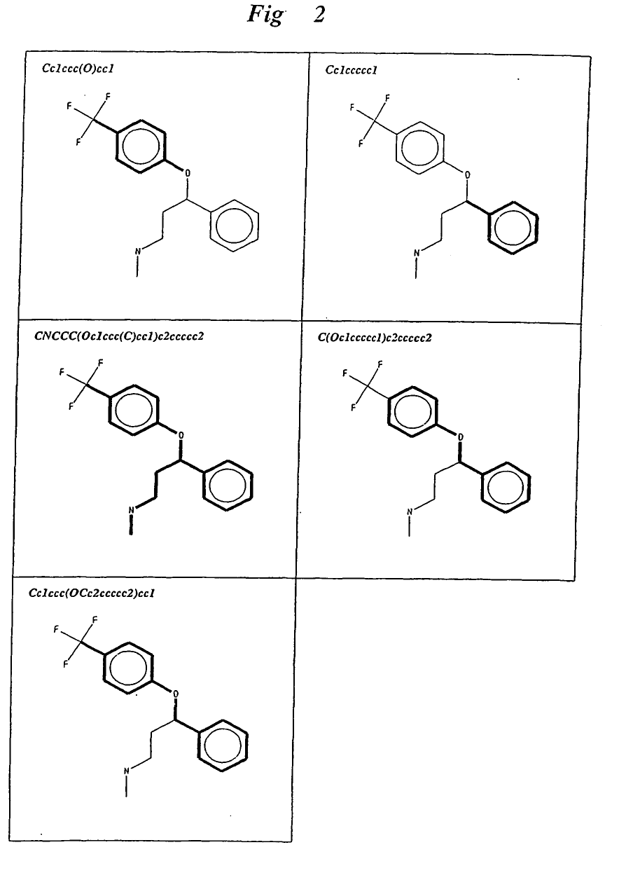

FIG. 1 shows a chart of the chemical structures of a total of 79 different compounds which are already known to be pharmaceutically active,

FIG. 2 shows examples of fragmentation patterns,

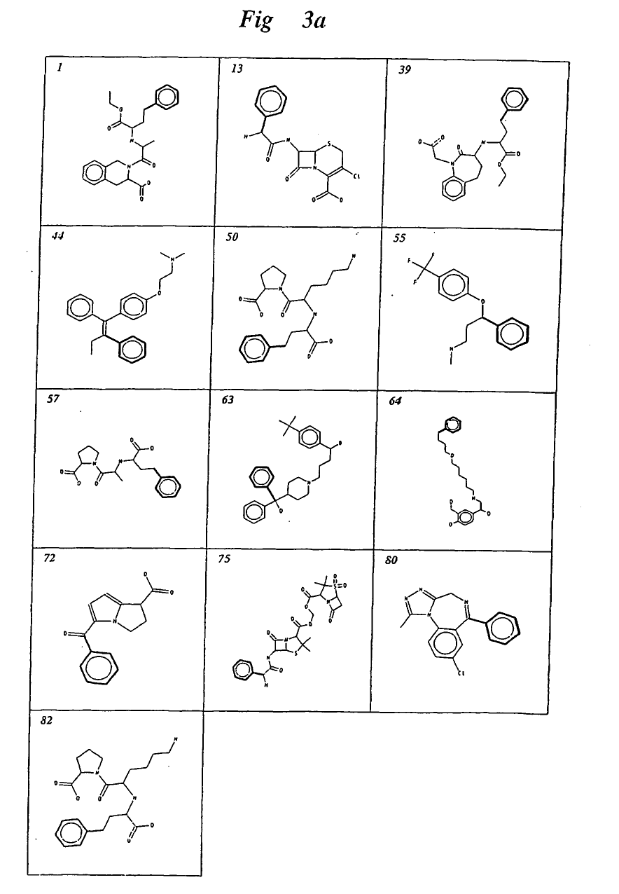

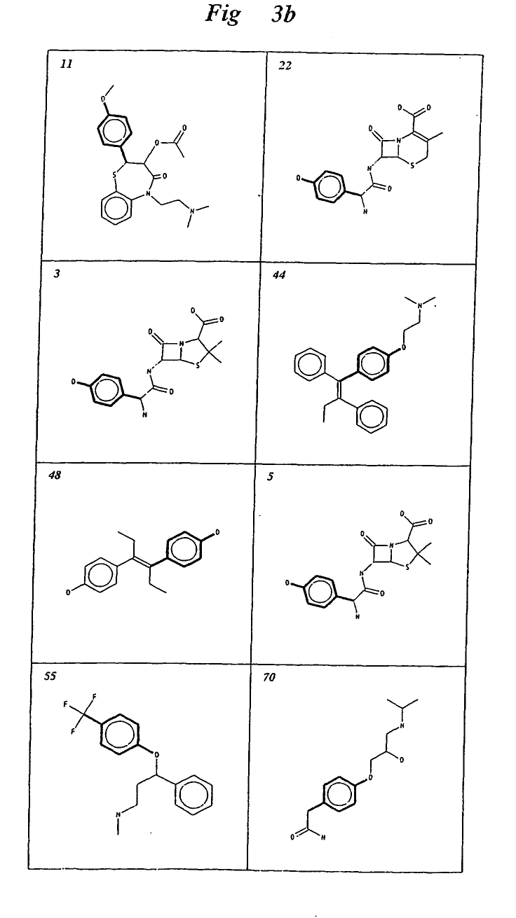

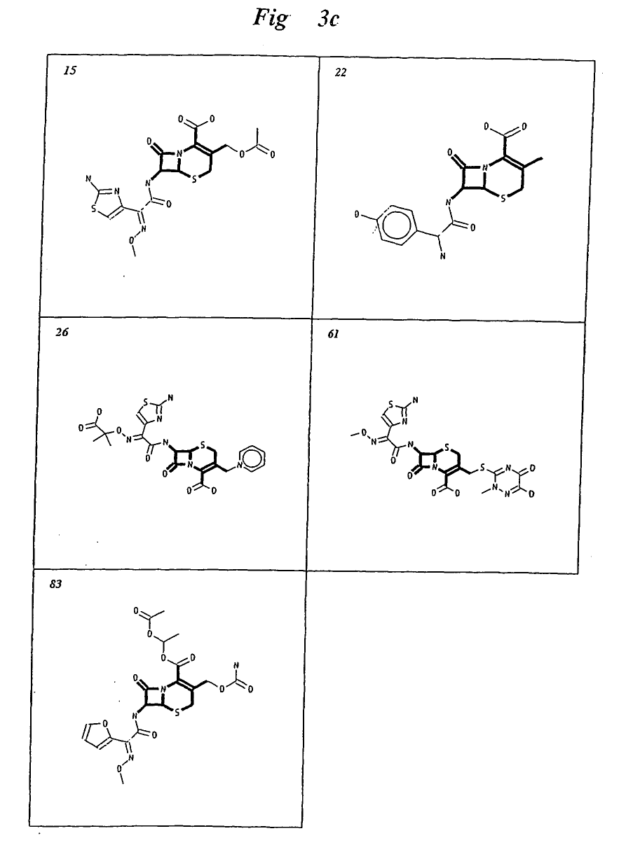

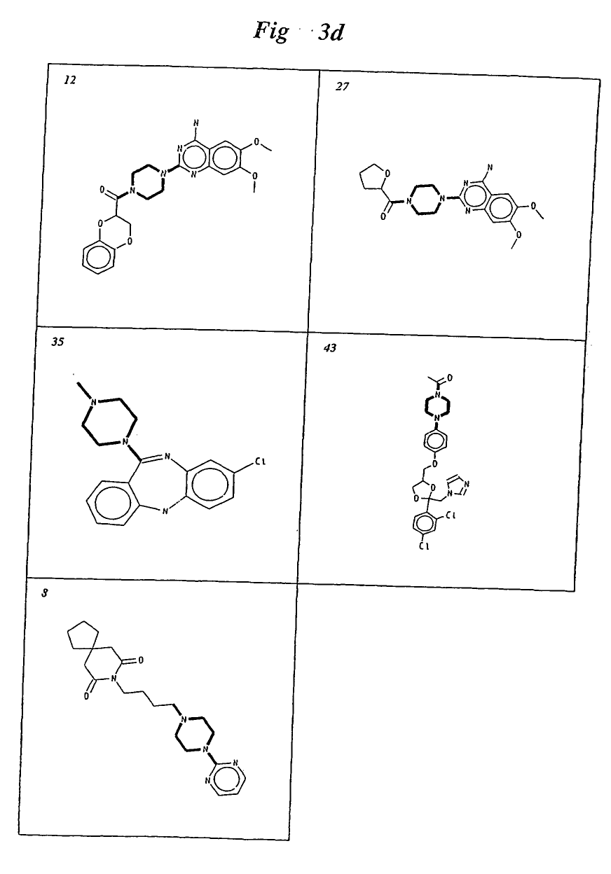

FIG. 3a shows a group of structures which in each case have a common fragment,

FIGS. 3b-d show three further groups of substances which have common structural fragments, and

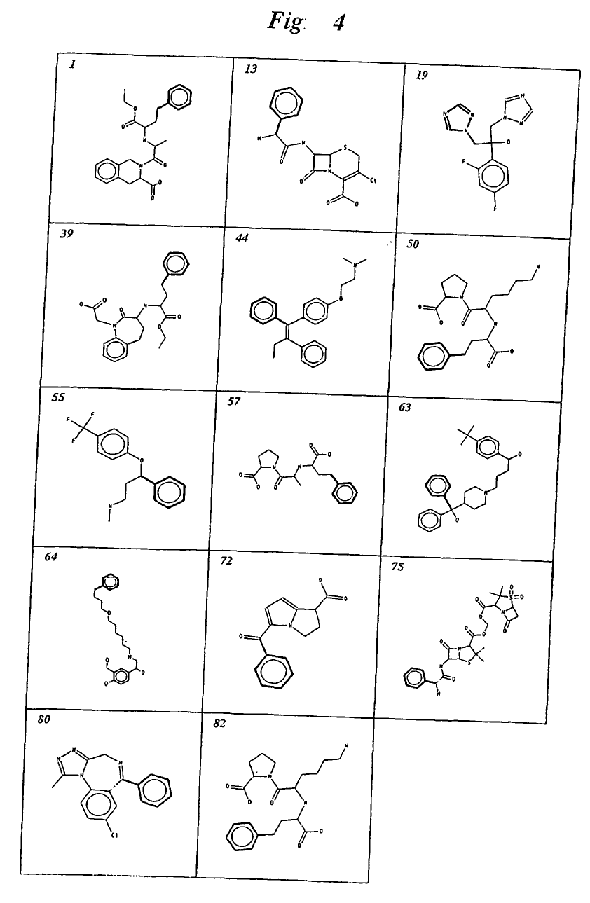

FIG. 4 shows the extension of the group according to FIG. 3a by an isosteric transformation (inclusion of the substance denoted by 19).

Table 2 shown below contains all names and structures of a use example of the present invention. The SMILES code was used for the linearized representation of the structures. The use example comprises a total of 79 compounds which are already known to be pharmaceutically active and for which it is to be assumed here that they correspond to a hit list from an HTS experiment.

In FIG. 1, these compounds characterized by a serial number in Table 2 are shown graphically in the form of their chemical structure.

FIG. 2 shows different structural elements of the compound 52, each of which could serve as fragment for characterizing a group of substances. The fragment shown in the upper right quadrant of FIG. 2 and emphasized in bold print serves, for example, as a characteristic element of the group which is shown in FIG. 3a. All structures which are reproduced there and which can also be identified on the basis of their numbers in the overview according to FIG. 1 and Table 2 have exactly this one element. The element which can be seen in the upper left quadrant of FIG. 2 serves for forming the group according to FIG. 3b. It should be pointed out that the substance with the number 55 belongs to both groups according to FIG. 3a and according to FIG. 3b, since both fragments are present in this substance. As can be seen in the upper two quadrants of FIG. 2, the two fragments mentioned were also formed precisely by fragmentation of one and the same substance No. 55.

FIG. 3c shows a further fragment which is emphasized by bold print and which is common to five different substances of the use example, which in turn are characterized by their numbers. Finally, FIG. 3d also shows a fragment which appears very similar to the fragment forming the group according to FIG. 3b, except that, instead of two diametrically opposite carbon atoms, the ring is completed by two nitrogen atoms from which two single bonds also emanate. It would accordingly be entirely possible to combine the groups according to FIGS. 3b and 3d into one group by applying this similarity consideration.

In FIG. 4, the fragment which was used for forming the group from FIG. 3a was once again taken as a basis, in this case, on the basis of a bioisosteric transformation described in the literature, the substance characterized by the number 19 also being added, which substance does not have the relevant fragment in identical form but has one in which the characteristic benzene ring of this fragment has been exchanged for an aromatic five-membered ring having three nitrogen atoms.

A comparative consideration of the groups gives the following results:

The following molecules form groups having at least 5 members:

Group 1: 1,13,39,44,50,55,57,63,64,72,75,80,82

Group 2: 3,5,11,22,44,48,55,70

Group 3: 15,22,26,61,83

Group 4: 8,12,27,5,43

Groups 1 and 2 have the compounds 44 and 55 in common, groups 2 and 3 both contain the compound 22. For group 4, there is no relationship with the other groups. The constituent fragment of group 1 is a partial structure of the corresponding fragment of group 2.

A form which permits a rapid overview of structure-function relationships of the respective data is thus available. The user of the system according to the invention expediently makes use of this from a computer workstation having appropriate equipment so that the prepared data serves for rapid and comprehensive orientation of the researcher.

The inclusion of a rule involving bioisosteric transformation can be effected at the level of the fragments produced, since there is existing medical-chemical knowledge in published form which expresses the equivalence of molecular fragments with respect to specific biological/pharmacological action. It must be borne in mind that the relations found are not necessarily generally applicable. The use of equivalence relations of fragments leads, in the method of the present invention, to the combination of all lists which show these fragments, so that extended groups can now be detected. The extended group shown in FIG. 4 shows a relationship on the basis of the test data set used. Molecule 5 is additionally introduced in group 1 since there is a biosteric relationship between the methyltriazole radical and the phenyl radical. Because of this result, the number of common members of groups 1 and 2 increases.

A major advantage of the present invention is that such equivalence relations can be recognized from the actual HTS data so that this knowledge is then available for further evaluations of experimental data. Consequently, the present invention permits specific learning on the basis of the experiments performed.

In the case of a larger number of hits, as are to be expected in the case of a realistic HTS experiment, the number of possible groups and the number of individual group members do of course also increase compared with the example presented here. Nevertheless, it is however recognized that the individual groups which are shown in FIGS. 3a and 3d are numerically substantially more comprehensible than the totality of the hits which are shown in Table 2 and FIG. 1. By appropriate evaluation of these groups, one or other group can possibly also be excluded, so that in the end the number of substances to be investigated in more detail is concentrated into two or three groups and hence in an acceptable quantity.

For the evaluation of the substances or groups of substances, a vector with descriptors is now generated for each compound considered. Table 1 gives examples of substance properties of the present invention which are used.

Each individual property is evaluated using different mathematical functions and empirically determined rules and “property scores” are calculated. Suitable scoring functions are also the arc tangent, the arc cotangent, the hyperbolic tangent and the area sine function. Functions of the type y=b exp(−ax²) and y=a exp(bx+cx²) can also be used.

The functions contain a number of parameters, for example the base of the exponential functions, slopes and points of inflection, which are determined with the aid of QSAR methods empirically for each substance property.

If specific properties are not available for molecules, the scoring function can be modified so that, for example, a very large value (e.g. 1000) is assigned to the lacking property in the property score. This approach makes it possible to complete the property profile in a specific manner only for the relevant compounds.

The evaluation of the property scoring is performed with the aid of the functions:

1. Cut-off function

This is the number of property scores outside/inside the optimum property ranges (principal errors, strengths of the substance). The list obtained can be sorted according to this cut-off value.

2. Total score function

The individual property scores are multiplied by a weighting factor and are summed/multiplied to give the total score.

$$Total score = {\sum }_{i = 1}^{n} {a}_{i} \cdot {Score}_{i} \begin{matrix}{a}_{i} & Weighting factors \\ {Score}_{i} & Property scores\end{matrix}$$

With the aid of the weighting factors, substance properties can be excluded from the property profile.

In addition, a balanced ratio of, for example, receptor properties, physicochemical and metabolic properties and/or also in vitro/in vivo properties can be specified. If sorting is performed by the total score, desired profiles can be specifically filtered out for the drug in this way.

The scoring scenario used is dependent on the specific experiment since the relevant properties are selected accordingly in each case. Moreover, the procedure can be adapted to the available number of molecules considered. In cases of a large number of HTS hits, the approach can be implemented as strict filtering whereas, where a small number of molecules are considered, it can be used for working out small, significant differences in the property space covered.

The scoring method can be used for eliminating substances having a suboptimal property profile from a substance pool. Thus, substances having advantageous properties can be selected from external sources for the substance pool. It is suitable for evaluating hit lists from HTS and for designing combinatorial or virtual libraries. It can also be used for finding new guidelines; and it can be employed for selecting substances for animal experiments or pharmacokinetic investigations. It is also suitable for lead optimization.

In the groups thus selected, the probability of success of finding a pharmaceutically valuable control molecule is considerably improved compared with the conventional procedures.

Without further elaboration, it is believed that one skilled in the art can, using the preceding description, utilize the present invention to its fullest extent. The preferred specific embodiments and examples are, therefore, to be construed as merely illustrative, and not limitative of the disclosure in any way whatsoever.

The entire disclosure of all applications, patents, and publications cited above and below, and of the corresponding application DE 101 08 590.7, filed Feb. 22, 2001, are hereby incorporated by reference.

TABLE 2
Names and structures (SMILES code) of the us xampl

| Ser. | Name | Structur |
|---|---|---|
| 1: | Accupril | CCOC(═O)C(CCc1ccccc1)NC(C)C(═O)N2Cc3ccccc3CC2C(═O)O |
| 2: | Adalat_line | COC(═O)C1═C(C)NC(═C(C1c2ccccc2[N+](═O)[O−])C(═O)OC)C |
| 3: | Amoxil | CC1(C)SC2C(NC(═O)C(N)c3ccc(O)cc3)C(═O)N2C1C(═O)O |
| 4: | Ativan_line | OC1N═C(c2ccccc2Cl)c3cc(Cl)ccc3NC1═O |
| 5: | Augmentin | CNC(═C[N+](═O)[O−])NCCSCc1csc(CN(C)C)n1 |
| 7: | Becotide/Beclovent | CC1CC2C3CCC4═CC(═O)C═CC4(C)C3(Cl)C(O)CC2(C)C1(O)C(═O)CO |
| 8: | Buspar | O═C1CC2(CCCC2)CC(═O)N1CCCCN3CCN(CC3)c4ncccn4 |
| 9: | Calan_line | COc1ccc(CCN(C)CCCC(C#N)(C(C)C)c2ccc(OC)c(OC)c2)cc1OC |
| 10: | Capoten | CC(CS)C(═O)N1CCCC1C(═O)O |
| 11: | Cardizem_line | COc1ccc(cc1)C2Sc3ccccc3N(CCN(C)C)C(═O)C2OC(═O)C |
| 12: | Cardura | COc1cc2nc(nc(N)c2cc1OC)N3CCN(CC3)C(═O)C4COc5ccccc5O4 |
| 13: | Ceclor_line | NC(C(═O)NC1C2SCC(═C(N2C1═O)C(═O)O)Cl)c3ccccc3 |
| 14: | Ciproxin_line | OC(═O)c1cn(C2CC2)c3cc(N4CCNCC4)c(F)cc3c1═O |
| 15: | Claforan | CON═C(C(═O)NC1C2SCC(═C(N2C1═O)C(═O)[O−])COC(═O)C)c3csc(N)n3 |
| 16: | Claritin_line | COC(═O)N1CCC(═C2c3ccc(Cl)cc3CCc4cccnc24)CC1 |
| 17: | Dalacin/Cleocin | CCCC1CC(N(C)C1)C(═O)NC(C(C)Cl)C2OC(SC)C(O)C(O)C2O |
| 18: | Depakote | CCCC(CCC)C(═O)[O−] |
| 19: | Diflucan | OC(Cn1cncn1)(Cn2cncn2)c3ccc(F)cc3F |
| 20: | Diprivan | CC(C)c1cccc(C(C)C)c1O |
| 21: | Dormicum/Versed | Cc1ncc2CN═C(c3ccccc3F)c4cc(Cl)ccc4n12 |
| 22: | Duricef | CC1═C(N2C(SC1)C(NC(═O)C(N)c3ccc(O)cc3)C2═O)C(═O)O |
| 23: | Estraderm | CC12CCC3C(CCc4cc(O)ccc34)C2CCC1O |
| 24: | Eulexin | CC(C)C(═O)Nc1ccc([N+](═O)[O−])c(c1)C(F)(F)F |
| 25: | Feldene | CN1C(═C(O)c2ccccc2S1(═O)═O)C(═O)Nc3ccccn3 |
| 26: | Fortum/Fortaz | CC(C)(ON═C(C(═O)NC1C2SCC(═C(N2C1═O)C(═O)[O])C[n+]3ccccc3)c4csc(N)n4)C(═O)O |
| 27: | Hytrin | COc1cc2nc(nc(N)c2cc1OC)N3CCN(CC3)C(═O)C4CCCO4 |
| 28: | Imigran/Imitrex | CNS(═O)(═O)Cc1ccc2[nH]cc(CCN(C)C)c2c1 |
| 29: | Intal | OC(COc1cccc2cc(cc(═O)c12)C(═O)[O−])COc3cccc4oc(cc(═O)c34)C(═O)[O−] |
| 30: | Istin/Norvasc | CCOC(═O)C1═C(COCCN)NC(═C(C1c2ccccc2Cl)C(═O)OC)C |
| 31: | Klonopin | [O−][N+](═O)c1ccc2NC(═O)CN═C(c3ccccc3Cl)c2c1 |
| 32: | Lamisil | CN(CC═CC#CC(C)(C)C)Cc1cccc2ccccc12 |
| 33: | Lasix | NS(═O)(═O)c1cc(C(═O)O)c(NCc2ccco2)cc1Cl |
| 34: | Leponex/Clozaril | CN1CCN(CC1)C2═Nc3cc(Cl)ccc3Nc4ccccc24 |
| 35: | Lipostat/Pravachol | CCC(C)C(═O)OC1CC(O)C═C2C═CC(C)C(CCC(O)CC(O)CC(═O)[O−])C12 |
| 36: | Lodine | CCc1cccc2c3CCOC(CC)(CC(═O)O)c3[nH]c12 |
| 37: | Losec/Prilosec | COc1ccc2[nH]c(nc2c1)S(═O)Cc3ncc(C)c(OC)c3C |
| 38: | Lotensin | CCOC(═O)C(CCc1ccccc1)NC2CCc3ccccc3N(CC(═O)O)C2═O |
| 39: | Lustral/Zoloft | CNC1CCC(c2ccc(Cl)c(Cl)c2)c3ccccc13 |
| 40: | Mevacor | CCC(C)C(═O)OC1CC(C)C═C2C═CC(C)C(CCC3CC(O)CC(═O)O3)C12 |
| 41: | Nizoral | CC(═O)N1CCN(CC1)c2ccc(OCC3COC(Cn4ccnc4)(O3)c5ccc(Cl)cc5Cl)cc2 |
| 42: | Nolvadex | CCC(═C(c1ccccc1)c2ccc(OCCN(C)C)cc2)c3ccccc3 |
| 43: | Paraplatin | O═C1O[Pt]OC(═O)C21CCC2 |
| 44: | Pepcid | NC(═Nc1nc(CSCCC(═NS(═O)(═O)N)N)cs1)N |
| 45: | Premarin_line | CCC(═C(CC)c1ccc(O)cc1)c2ccc(O)cc2 |
| 46: | Prepulsid/Propulsid | COC1CN(CCCOc2ccc(F)cc2)CCC1NC(═O)c3cc(Cl)c(N)cc3OC |
| 47: | Prinivil | NCCCCC(NC(CCc1ccccc1)C(═O)O)C(═O)N2CCCC2C(═O)O |
| 48: | Procardia_line | COC(═O)C1═C(C)NC(═C(C1c2ccccc2[N+](═O)[O−])C(═O)OC)C |
| 49: | Proscar | CC(C)(C)NC(═O)C1CCC2C3CCC4NC(═O)C═CC4(C)C3CCC12C |
| 50: | Proventil | CC(C)(C)NCC(O)c1ccc(O)c(CO)c1 |
| 51: | Provera_line | CC1CC2C3CCC(O)(C(═O)C)C3(C)CCC2C4(C)CCC(═O)C═C14 |
| 52: | Prozac | CNCCC(Oc1ccc(cc1)C(F)(F)F)c2ccccc2 |
| 53: | Relifex/Relafen | COc1ccc2cc(CCC(═O)C)ccc2c1 |
| 54: | Renitec/Vasotec | CC(NC(CCc1ccccc1)C(═O)O)C(═O)N2CCCC2C(═O)O |
| 55: | Retrovir | Cc1cn(C2CC(N═[N+]═[N−])C(CO)O2)c(═O)[nH]c1═O |
| 56: | Risperdal | Cc1nc2CCCCn2c(═O)c1CCN3CCC(CC3)c4noc5cc(F)ccc45 |
| 57: | Roaccutane/Accutane | CC(═CC═CC(═CC(═O)O)C)C═CC1═C(C)CCCC1(C)C |
| 58: | Rocephin | CON═C(C(═O)NC1C2SCC(═C(N2C1═O)C(═O)[O−])CSc3nc(═O)c([O−])nn3C)c4csc(N)n4 |
| 59: | Seldane_line | CC(C)(C)c1ccc(cc1)C(O)CCCN2CCC(CC2)C(O)(c3ccccc3)c4ccccc4 |
| 60: | Serevent | OCc1cc(ccc1O)C(O)CNCCCCCCOCCCCc2ccccc2 |
| 61: | Seroxat/Paxil | Fc1ccc(cc1)C2CCNCC2COc3ccc4OCOc4c3 |
| 62: | Tagamet | CNC(═NC#N)NCCSCc1nc[nH]c1C |
| 63: | Tegretol | NC(═O)N1c2ccccc2C═Cc3ccccC13 |
| 64: | Tenormin | CC(C)NCC(O)COc1ccc(CC(═O)N)cc1 |
| 65: | Timoptic_line | CC(C)(C)NCC(O)COc1nsnc1N2CCOCC2 |
| 66: | Toradol | OC(═O)C1CCn2c(ccc12)C(═O)c3ccccc3 |
| 67: | Transderm-Nitro | [O−][N+](═O)OCC(CO[N+](═O)[O−])O[N+](═O)[O−] |
| 68: | Trental | CC(═O)CCCCn1c(═O)n(C)c2ncn(C)c2c1═O |
| 69: | Unasyn | CC1(C)SC2C(NC(═O)C(N)c3ccccc3)C(═O)N2C1C(═O)OCOC(═O)C4N5C(CC5═O)S(═O)(═O)C4(C)C |
| 70: | Vancenase/Vanceril | CCC(═O)OCC(═O)C1(OC(═O)CC)C(C)CC2C3CCC4═CC(═O)C═CC4(C)C3(Cl)C(O)CC21C |
| 71: | Ventolin | CC(C)(C)NCC(O)c1ccc(O)c(CO)c1 |
| 72: | Voltaren | OC(═O)Cc1ccccc1Nc2c(Cl)cccc2Cl |
| 73: | Xanax/Alprazolam | Cc1nnc2CN═C(c3ccccc3)c4cc(Cl)ccc4n12 |
| 74: | Zantac | CNC(═C[N+](═O)[O−])NCCSCc1ccc(CN(C)C)o1 |
| 75: | Zestril | NCCCCC(NC(CCc1ccccc1)C(═O)O)C(═O)N2CCCC2C(═O)O |
| 76: | Zinnat/Ceftin | CON═C(C(═O)NC1C2SCC(═C(N2C1═O)C(═O)OC(C)OC(═O)C)COC(═O)N)c3ccco3 |
| 77: | Zocor | CCC(C)(C)C(═O)OC1CC(C)C═C2C═CC(C)C(CCC3CC(O)CC(═O)O3)C12 |
| 78: | Zofran | Cc1nccn1CC2CCc3c(C2═O)c4ccccc4n3C |
| 79: | Zovirax | Nc1nc(O)c2ncn(COCCO)c2n1 |

1. Method for determining pharmaceutically active substances with the aid of biological test series, with which the action of a large number of different substances on one or more biological targets is tested and a hit list of those substances prepared which actually or apparently have shown a pharmaceutical action on at least one biological target, whereupon a further selection is made among these hits for more detailed investigation of the pharmaceutical activity and/or applicability, characterized by the following features:
a) Determination of the chemical structure of the substances in the hit list,
b) splitting of the structure of the substances into individual components or fragments,
c) classification of the substance into groups having in each case identical and/or virtually identical or similar fragments,
d) evaluation of the pharmacological activity of the various groups,
e) comparison of the pharmacological activity of the various groups and
f) selection of substances from those groups which, on the basis of the evaluation and of the comparison according to steps d and e, have the highest activity.

2. Method according to claim 1, characterized in that the chemical structure is expressed in a linearized form.

3. Method according to either of claims 1 and 2, characterized in that the analysis of the structure for determining structural fragments comprises at least some of the following steps:
a) Removal of all simple substitutions on a molecular skeleton,
b) extraction of the associated ring systems of a molecule,
c) extraction of all simple ring structures of a molecule,
d) extraction of all molecular moieties present in the form of a chain,
e) selection of topological paths within a molecule with predetermined length,
f) concentric atomic neighbourhood spheres around selected atoms,
g) combination or permutation of all substituents of a molecular skeleton,
h) determination of the sets of atoms linked via topological paths,
i) fragmentation by breaking of bonds of defined chemical groups,
j) sets of torsional angles of atoms, and
k) existing predefined functional groups.

4. Method according to any of claims 1 to 3, characterized in that the evaluation of the pharmacological activity according to step d) is effected by the individual evaluation of the individual substances and combination of the individual evaluations of all substances (members) of a group under a specifiable first set of criteria which comprises at least one or more of the following parameters:
Molecular weight, molecular size, ionization, basicity, acidity, lipophilicity/amphiphilicity, solubility, octanol/water partition coefficient, number of hydrogen bridge acceptors and donors, stability in gastric and intestinal fluid, permeability values for various cell types and toxicity.

5. Method according to any of claims 1 to 4, characterized in that the evaluation parameters are expressed as numerical values by standardized functions.

6. Method according to claim 5, characterized in that in general different weighting functions are assigned to the different properties.

7. Method according to any of claims 1 to 6, characterized in that bioisosteric substances are additionally included in those groups of substances which are characterized by identical fragments.

8. Method according to any of claims 1 to 7, characterized in that the groups of substances which are defined by a common fragment are supplemented by further substances of the test series which have the fragment characterizing the group and which have shown no pharmacological activity in the test series.

9. Method according to any of claims 1 to 8, characterized in that groups whose number of active members is below a specified limit are excluded from the further investigation.

10. Method according to any of claims 1 to 9, characterized in that groups whose members have an average evaluation below a specified limit are excluded from the further investigation.

11. Method according to any of claims 1 to 10, characterized in that the evaluation parameters (x) in the form
$${S}_{x} = \frac{x - {x}_{min}}{{x}_{max} - {x}_{min}}$$
are used as molecular values for the evaluation of the substances.

12. Method according to any of claims 1 to 11, characterized in that the structural data and the evaluation parameters of the substances are stored in digital form.
